(19) United States
(12) Patent Application Publication
(10) Pub. No.: US 2013/0045270 A1
(43) Pub. Date: Feb. 21, 2013

(54) METHOD OF USE OF STABILIZED GROWTH FACTOR IN SKIN CARE

(71) Applicants: Einar Mantyla, Reykjavik (IS); Bjorn Larus Orvar, Kopavogur (IS); Hakon Orn Birgisson, Reykjavik (IS)

(72) Inventors: Einar Mantyla, Reykjavik (IS); Bjorn Larus Orvar, Kopavogur (IS); Hakon Orn Birgisson, Reykjavik (IS)

(21) Appl. No.: 13/542,807

(22) Filed: Jul. 6, 2012

(30) Foreign Application Priority Data
Jul. 6, 2011 (IS) 8968

(51) Int. Cl. A61K 38/18; A61P 17/10; A61K 9/127; A61Q 19/02; C07K 14/485; A61P 17/02; A61K 8/64; A61P 17/00; A61P 31/10

(52) U.S. Cl. 424/450; 514/9.6; 530/300; 424/62

(57) ABSTRACT

Dermatologic compositions for therapeutic use, containing a growth factor, or a growth factor purified from transgenic plants, or animal, bacterial, yeast or insect cells, or a mixture of growth factors in purified form, for treatment of various dermatological conditions and diseases. The pharmaceutical compositions are suitable for treatment of sensitive and challenged skin due to conditions and illnesses such as challenged skin due to systemic lupus erythematosus, Lichen simplex chronicus, blepharitis and blepharokeratoconjuctivitis. The skin compositions are also useful for use in combination with agents to treat anti-fungal infections such as Clotrimazole, Terbinafine, Miconazole, Ketoconazole, Amorolfine and Econazole.

### FIELD OF THE INVENTION

The present invention generally relates to dermatological compositions comprising stabilized growth factors and cytokines for skin care, dermatological applications and methods for making dermatological pharmaceutical products. In particular, this invention relates to stabilized heterologous growth factors which are preferably obtained from transgenic or transiently expressing plants or isolated from a suitable host organism or cell expressing the heterologous protein, such as bacterial, yeast, animal cells including insect cells, and their use in pharmaceutical products.

### BACKGROUND OF THE INVENTION

Skin is the biggest organ of the human body carrying out various functions such as protection, barrier, temperature controlling, excretion and respiration. With time and ageing, those functions rapidly decline, and a variety of physiological changes occur to the skin. These changes are manifested in the decrease in the thickness of epidermis, dermis and subcutaneous tissue, which are the main components of skin. Changes in lipid composition undermine the moisture barrier role of lipid layers and resulting in the dryness of skin. Further, with age, the occurrence of age spots, freckles, pigmentation or various skin lesions also increases.

An age-dependent decrease in epidermal turnover rate is involved in accumulation of low quality Stratum Corneum, resulting in senile xerosis, undue pigmentation and fine wrinkles. This may, in part, be due to aberrant keratinocyte differentiation.

Environmental components such as pollution and UV-rays, can speed up the ageing of the skin. Reactive oxygen species and free radicals and some physiological states such as fatigue or stress are particularly detrimental to proteins, nucleic acids and membrane lipids, leading to the aging of the skin. Accordingly, there have been many studies on the occurrence of the wrinkles, age spots or freckles, the loss of skin elasticity, the pigmentation, and the dryness and cracking of skin.

### Skin Conditions:

Skin and epidermis is frequently damaged due to exposure to radiation, resulting in reduced elasticity and strength of the epidermis, discoloration and increased scarring. Radiation damages to skin may be caused by sunburns (excessive exposure to UV radiation), radiotherapy, as in cancer treatments, and exposure to harmful x-ray and/or gamma ray radiation from radioactive material.

Skin may become damaged due to contact with chemicals or substances that induce photosensitation of the skin resulting in discoloration and epidermal.

Exposure to excessive or damaging radiation of normal skin or photosensitized skin may result in abnormal pigmentation (melasma) that can cause discomfort, suffering and aesthetic concerns affecting quality of life.

Growth factors are key players in regulating proliferation and differentiation of cells and are involved in restructuring the epidermis and basal lamina upon injury or damage. They are important for the renewal of cells and thus, can counteract several aspects of aging and normalize keratinocyte differentiation, fibroblast growth and induce turnover and renewal of cells and cellular products.

It is recognized that growth factors can have beneficial effects on various skin disorders and skin injuries and counteract effects of aging that are the result of impaired or deteriorating protective mechanisms at cellular level. Growth factors can promote cellular renewal and proliferation and are a natural component of the healing process of wounds.

The Epidermal Growth Factor (EGF) promotes the division of various epithelial cells originated in the ectoderm and mesoderm. It is extensively distributed in body fluid, especially in urine and breast milk (Carpenter, G. and Cohen, S., “Epidermal growth factor,” Ann Rev. Biochem., 48, 192-216 (1979)). It is a single polypeptide consisting of 53 amino acid residues and has a molecular weight of 6,200 Daltons (Campion, S. R. and Niyogi, S. K., “Interaction of epidermal growth factor with its receptor”). In 1962, Cohen isolated EGF from the gland beneath the chin of the mature male mouse. In 1972, Savage and Taylor identified the primary structure of mouse EGF and the location of three intramolecular disulfide bonds in EGF that are essential for physiological function.

EGF is believed to have an excellent effect on skin injuries because it strongly promotes the proliferation of epithelial cells, endothelial cells and fibroblasts, and also the migration and proliferation of epithelial cells to where they are deficient. Growth factors are key players in maintenance of tissue integrity and in cell to cell communication, thus playing a protective role in fighting degeneration of epidermal tissue.

U.S. Pat. No. 5,618,544 incorporated herein by reference in its entirety, discloses a cosmetic composition comprising EGF, TGF-a and FGF for decreasing cutaneous senescence and improving the appearance of skin.

U.S. Pat. No. 6,589,540 teaches that EGF remarkably enhances the effect of retinol used in skin care products, and also effectively alleviates the skin irritation of retinol.

U.S. Pat. No. 7,517,528 teaches a method and composition for treating skin wounds with EGF.

U.S. Pat. No. 7,799,760 teaches that injecting EGF into diabetic ulcers may prevent amputation.

Growth factors such as PDGF are released at wound site during coagulation phase, and act as chemo-attractants for neutrophils, macrophages and fibroblasts. These cells play an important role in killing bacteria and removal of necrotic debris at the wound site. Activated macrophages release in turn growth factors that promote angiogenesis and communicate with the B-cell and T-cell mediated immune responses. Macrophages secrete TGF-beta, that stimulates fibroblasts to produce new extracellular matrix, and VEGFs that stimulate angiogenesis. Epithelization proceeds as keratinocytes divide and cover the wound bed. Thus, it is well established that growth factors are important mediators of healing process and studies indicate that G-CSF may be beneficial for treating infected diabetic ulcerations. EGF stimulates the proliferation of fibroblasts and keratinocytes.

FGF has proliferative effects on epithelial cells and has been observed to accelerate bone and wound healing in animal models. KGF-2 accelerates wound healing significantly, especially the closing of wounds.

### SUMMARY OF THE INVENTION

It is an object of the present invention to provide growth factors for specific clinical use for sensitive and challenged skin due to conditions and illnesses stated here below.

In one aspect of the present invention, stabilized growth factors can be used with positive mitigating effects on challenged skin due to systemic lupus erythematosus, Lichen simplex chronicus, blepharitis and blepharokeratoconjuctivitis.

Lichen simplex chronicus, also called neurodermatitis, is a common skin problem. It generally affects adults, and may result in one, or many itchy patches.

Lichen simplex chronicus is a type of dermatitis, and is usually the result of repeated rubbing or scratching. The stimulus to scratch may be unrecognized, it may result from a mosquito bite, stress, or simply a nervous habit. The result is a very itchy patch of skin, often located on the nape of the neck, the scalp, the shoulder, the wrist, or the ankle. The affected skin is thickened, often appearing as a group of small firm papules (bumps). The skin markings are more visible, and the hairs are often broken-off. The ithcing and scratching can become habitual and the cycle of chronic itching and scratching can cause the affected skin to become thick and leathery.

It is an object of the invention to effectively mitigate the symptoms of Lichen simplex chronicus, relief from the itching and alleviation of skin markings, thus, breaking the vicious cycle of itching and scratching.

It is an aspect of the invention to provide a useful composition to mitigate symptoms of nickel allergy that may cause or aggravate abnormal skin conditions manifested as blisters, scaling of skin and/or lacerations.

It is further an important aspect of the invention to provide composition with growth factor with positive effect on challenged or damaged skin that has been exposed to irritating or damaging conditions due to radiation or chemicals resulting in photosensitation of the skin, such as psoralens and furocoumarins.

Such damages may be resulting from radiotherapy treatment or exposure to radiation, x-ray and/or gamma radiation or excessive UV-radiation. The growth factor based composition of the present invention results in speeding up revitalisation of the damaged epidermis and partial or full restoration of normal epidermis and skin.

In another aspect of the present invention growth factor-containing compositions are provided which are effective against abnormal pigmentation of the skin; melasma or chloasma and postinflammatory hyperpigmentation. The compositions reduce the pigmentation and results in more even visual appearance of the skin.

A suitable growth factor or combination of two or more growth factors for the invention may be selected from recombinant growth factors including but not limited to Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g), Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2, BMP-3, BMP-4, BMP-5, BMP-6, BMP-7, BMP-8a), Hepatocyte Growth Factor (HGF), Leptin, Noggin, and Thymosin beta 4. These growth factors may be used according to the invention in healing of inflicted, pathological and surgical wounds and reduction/prevention of scar tissue formation. A selection of growth factors may be used for an ex vivo treatment in an operation such as hair-transplant, e.g. by immersing the excised follicle units in a solution containing plant-derived recombinant growth factors to improve viability of the excised follicle units and to speed up and progress the healing process following the transplantation.

A choice of growth factors such as thymosin beta 4 and noggin are examples of preferred growth factors for this use of the invention, they are found to disrupt a refractive stage of cells in hair follicules at post-transplantational stage, and induce hair growth. The present invention provides compositions and means to treat scalp and follicles and/or follicle units (FU) in refractive stage with safe plant-derived human growth factors, in a hypoallergenic formula to revitalize hair growth and for healing from the effects of the transplantation surgery. It is a significant improvement in a number of therapeutic applications to be able to use recombinant human growth factors that are not sourced from human or animal source, and are not contaminated with bacterial endotoxins.

The present invention further provides growth factors for use in combination with antifungal agent therapy for treating fungal skin infections. The formulations of the invention as described above can be used with oral antifungal therapies or used in combination with topical antifungal therapies, or an antifungal agent can be included in the topical formulations of the invention. A suitable antifungal agent for use in combination with growth factor according to the invention to treat anti-fungal infections are agents comprising active substances selected from but not limited to Clotrimazole, Terbinafine, Miconazole, Ketoconazole, Amorolfine and Econazole. The present formulations are also suitable for use following anti-fungal therapy, to heal and recondition skin which is sensitive after the treatment.

It is an object of the invention to provide useful compositions containing growth factors to reduce stretch marks on skin caused by excessive tension to the epidermis, such as during pregnancy, obesity or mechanical tension applied to the skin. The present invention is useful to restore and improve the elasticity of the skin in the damaged area and thus reduce the signs of damage as in stretch marks.

In an important aspect of the invention a growth factor and composition effective against acne is provided. Acne can be classified into twenty categories, all of which are negatively affecting the complexion and appearance of the skin. Acne is often accompanied by localized inflammation and infection of the skin. A composition based on growth factors according to the invention is effective in reducing acne and restoring and improving the health of the skin, resulting in reduced inflammation and infection of the skin.

It is an object of this invention to present methods of use of stabilized heterologous recombinant growth factor in combination with an antifungal topical composition. Such a combination composition enables the topical use of growth factors to act as wound healing agents after the antifungal composition has terminated the fungal infection. Antifungal agents used in combination with growth factor containing composition may include antifungal agents, from the group of polyene antifungals, imidazole, triazole, thiazole antifungals and allylamin.

Further, the present invention provides the above mentioned growth factors for use as a medicament for any of the above stated conditions and ailments.

### BRIEF DESCRIPTION OF THE DRAWINGS

FIG. 1A is an image of skin of a subject diagnosed with a nickel allergy showing allergic contact dermatitis before treatment.

FIG. 1B is an image after six day treatment with an EGF growth factor-containing glycerol based composition.

FIG. 2 A is an image of skin of a subject diagnosed with a nickel allergy showing allergic contact dermatitis before treatment.

FIG. 2 B is an image after thirteen day treatment with an EGF growth factor-containing glycerol based composition.

FIG. 3 A is an image of skin of a subject diagnosed with a nickel allergy showing allergic contact dermatitis before treatment.

FIG. 3 B is an image after thirteen day treatment with an EGF growth factor-containing glycerol based composition.

FIG. 4 A is an image of skin of a subject diagnosed with Neurodermatitis before treatment.

FIG. 4 B is an image after four weeks treatment with an EGF growth factor composition.

FIG. 5 A is an image of skin of a subject diagnosed with Folliculitis before treatment.

FIG. 5 B is an image after one week treatment with an EGF growth factor composition.

FIG. 5 C is an image after twelve week treatment with an EGF growth factor composition.

FIG. 6 A is an image of skin of a subject with mild to moderate acne before treatment.

FIG. 6 B is an image after fourteen day treatment with an EGF growth factor composition.

### DETAILED DESCRIPTION OF THE INVENTION

As used herein, a “plant-derived” growth factor is a growth factor obtained (produced) from a transgenic plant or progenies of a transgenic plant, or growth factor transiently expressed in a plant and is used interchangeably with the term “plant-produced”. Thus, the term “plant-derived growth factor” refers in the context of the application generally to a heterologous growth factor, non-native to the host plant which is used as a production vehicle. The growth factor according to the present invention may be any human or non-human growth factor where its gene introduced into the plant or progenitors of the plant, preferably using recombinant technology. The isolated growth factor may be used as an active ingredient in a therapeutic topical composition. (The term human and non-human refers to the genetic origin/identity of the corresponding gene sequence that encodes the protein.)

In the present context the term “non-plant derived heterologous growth factor” refers to growth factors, preferably human growth factors, obtained (produced) from a suitable non-plant host organism expressing the heterologous protein, such as but not limited to bacterial, yeast, fungal, or animal cells, including insect cells.

Methods for introducing and expressing foreign genes in plants and other host organisms and cells are well known in the art. A plant that can be genetically transformed is a plant into which heterologous DNA sequence, including DNA sequence for a coding region, can be introduced, expressed, stably maintained, and transmitted to subsequent generations of progeny. Genetic manipulation and transformation methods have been used to produce barley plants that are using herbicide resistance including, for instance, bialaphos or basta, or antibiotic resistance, such as hygromycin resistance, as a selectable marker.

Suitable cultivars are selected and a suitable method for introduction of foreign gene selected. The term “transformation” or “genetic transformation” refers to the transfer of a nucleic acid molecule into the genome of a host organism, resulting in genetically stable inheritance. Host organisms containing the transformed nucleic acid fragments are referred to as “transgenic” organisms. A “transgenic plant host cell” of the invention contains at least one foreign, preferably two foreign nucleic acid molecule(s) stably integrated in the genome. Examples of methods of plant transformation include Agrobacterium-mediated transformation (De Blaere et al. 1987) and particle-bombardment or “gene gun” transformation technology (Klein et al. (1987); U.S. Pat. No. 4,945,050).

WO 2006/016381 describes a particular useful Barley cultivar amenable for transformation and describes in detail suitable transformation methods.

WO 2005/021762 discloses methods for modifying proteins by making chimeric proteins that are readily purified on a large scale.

Growth factors that are suitably produced and used according to the present invention may be selected from but are not limited to the species and groups including Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g), Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2, BMP-3, BMP-4, BMP-5, BMP-6, BMP-7, BMP-8a), Hepatocyte Growth Factor (HGF), Leptin, Noggin, and Thymosin beta 4.

In certain embodiments of the invention, the polypeptide of interest being produced in the transgenic plant contains an affinity tag at either N-terminal or C-terminal of the polypeptide, or at both ends. Such a tag may include repetitive HQ sequence, poly Histidine-tail, GST, CBM or any other useful affinity tag that simplifies purification of the heterologous peptide.

Hyaluronan is also called hyaluronic acid and hyaluronate, these terms are synonyms and interchangeable in the present context. Hyaluronan is an anionic, non-sulfated glycosaminoglycan distributed widely throughout connective, epithelial, and neural tissues.

The term skin care/dermatological composition as used herein encompasses both medical/pharmaceutical compositions for therapeutic dermatological applications as well as compositions for both for therapeutic and cosmetic use.

### Dosage

For topical therapeutic application in accordance with the invention, dose of growth factor is preferably in the range from 0.01 to 100 μg per gram of composition, and more preferably in the range 0.1 to 50 μg per gram. Compositions for the treatment of skin ageing or loss of hair preferably comprise from 0.2 to 50 μg of active substance per gram of composition.

The length of treatment varies depending on the pathology or on the desired effect. In the case of scleroderma treatment the application ranges from 1 day to 12 months according to the pathology severity. In the case of a treatment against natural or early ageing of the skin, the application ranges from 1 to 400 days, preferably for at least 30 days. Likewise, in the case of a treatment for preventing loss of hair or for promoting hair re-growth the application ranges from 1 to 400 days.

Dermatological compositions according to the invention can suitably be used for treatment of skin conditions including dry skin, eczema, dermatitis, rash, psoriasis, skin redness, and edema. Compositions of the invention are also useful for healing and reduction of scar tissue and healing and improving cracked skin on heels.

Preferably a transgenic plant extract is prepared from grains of barley containing any one or more of the proteins of the above listed growth factors, their mimetics or at least domains thereof that enable binding to, and activation of a growth factor receptor. The enclosed non-limiting examples show illustrative uses of different growth factors derived from transgenic barley extracts.

Extracts used according to the invention refer to protein extracts from the transgenic host plants, comprising the growth factor of interest. The growth factor can be only a minor component of the extract, provided that other proteins do not interfere with the activity of the growth factor or cause any other undesired effects. Such extracts are e.g. seed protein extracts from plants expressing the heterologous growth factor in their seeds. The extracts may also be purified to higher or lesser degree, i.e. they may be partially purified by one or more purification steps to enrich for the heterologous growth factor.

Numerous vehicles for topical application of pharmaceutical compositions are known in the art. See, e.g., Remington's Pharmaceutical Sciences, Gennaro, A. R., ed., 20th edition, 2000: Williams and Wilkins PA, USA. All compositions usually employed for topically administering pharmaceutical and cosmetic compositions may be used, e.g., creams, lotions, gels, dressings, shampoos, tinctures, pastes, serums, ointments, salves, powders, liquid or semiliquid formulation, patches, liposomal preparations, solutions, suspensions, liposome suspensions, W/O or O/W emulsions, pomades and pastes and the like as long as the heterologous protein as active ingredient is stabilized. Application of said compositions may, if appropriate, be by aerosol e.g. with a propellant such as nitrogen carbon dioxide, a freon, or without a propellant such as a pump spray, drops, lotions, or a semisolid such as a thickened composition which can be applied by a swab. In particular compositions, semisolid compositions such as salves, creams, lotions, pastes, gels, ointments and the like will conveniently be used.

The compositions of the invention can be provided for parenteral, systemic or local use, comprising solutions, suspensions, liposome suspensions, W/O (water/oil) or O/W (oil/water) emulsions. In a preferred embodiment the active substance is formulated in a lyophilized form, mixed to suitable lyophilization additives and ready to be redissolved with therapeutically acceptable diluents. Useful lyophilization additives are: buffers, polysaccharides, sucrose, mannitol, inositol, polypeptides, amino acids and any other additive compatible with the active substance. In a preferred embodiment of the invention the active substance is dissolved in phosphate buffer (NaH₂PO₄/H₂O—Na₂HPO₄/2H₂O) in an amount such that the post-lyophilization growth factor/phosphate ratio is comprised between 1:1 and 1:2. Diluents suitable for parenteral use are: water, physiological solutions, sugar solutions, hydroalcoholic solutions, oily diluents, polyols, like glycerol, ethylene or polypropylene glycol, or any other diluent compatible with the administration method as for sterility, pH, ionic strength and viscosity.

Preferably the vehicle of topical application is a formulation that is naturally anti-bacterial yet without any non-natural preservative or anti-microbial agent. It will be appreciated to use few ingredients and eliminate complex ingredients that may act as allergenics and/or irritants. The formulations should also ensure long term stability of the active protein ingredients, preferably providing long shelf life such as one year or longer at room temperature storage.

In a preferred embodiment the active compound, recombinant growth factor of choice, is added to a formulation suitable for topical application containing one or more of glycerol, a salt such as but not limited to sodium chloride, potassium chloride and calcium chloride, where calcium chloride is the most preferred, purified water, and ethanol, and preferably all of those. Such compositions are surprisingly shown to effectively stabilize the recombinant protein represented by the growth factor of choice. It is an aspect of the present invention that this formulation effectively stabilizes recombinant proteins whether or not the proteins are glycosylated. The formulation is preferably antibacterial by nature and therefore particularly suitable as a topical formulation for dermatological use. The composition of the invention may furthermore comprise an optional additive such as hyaluronic acid (hyalorunate).

In the case of emulsions or suspensions, the composition may contain suitable surfactants of non-ionic, zwitterionic, anionic or cathionic type commonly used in the formulation of medicaments. Oil/water (O/W) hydrophilic emulsions are preferable for parenteral systemic use, whereas water/oil (W/O) lipophilic emulsions are preferable for local or topic use.

Moreover, the compositions of the invention may contain optional additives like isotonic agents, such as sugars or polyalcohols, buffers, chelating agents, antioxidants, antibacterials.

Liquid forms according to the invention can comprise solutions or lotions. These may be aqueous, hydroalcoholic, like ethanol/water, or alcoholic and are obtained by solubilizing the lyophilized substance.

Alternatively, active substance solutions, may be formulated in form of gel by addition of known gelling agents, like: starch, glycerin, polyethylene or polypropylene glycol, poly(meth)acrylate, isopropyl alcohol, and hydroxystearate.

Other types of compositions for topic use are emulsions or suspensions in form of pomades, pastes, creams. W/O emulsions are preferable, providing a faster absorption. Examples of lipophilic excipients are: liquid paraffin, anhydrous lanolin, white vaseline, cetyl alcohol, stearyl alcohol, vegetable oils, mineral oils. Agents increasing cutaneous permeability, thereby facilitating the absorption, may advantageously be used. Examples of such agents are physiologically acceptable additives like polyvinyl alcohol, polyethylenglycol or dimethylsulfoxide (DMSO).

Other additives used in the topic compositions are isotonic agents, like sugars or polyalcohols, buffers, chelating agents, antioxidants, antibacterials, thickeners, dispersants.

It follows that the preparations may further contain conventional components usually employed in preparations described herein, including oils, fats, waxes, surfactants, humectants, thickening agents, antioxidants, viscosity stabilizers, chelating agents, buffers, preservatives, perfumes, dyestuffs, lower alkanols, and the like.

Delayed-release compositions for local or systemic use may be useful, and comprise polymers like polylactate, poly(meth)acrylate, polyvinylpyrrolidone, methylcellulose carboxymethylcellulose and other substances known in the art. Delayed-release compositions in form of subcutaneous implants based on, e.g. polylactate or other biodegradable polymers may be useful as well.

Though the active substance is preferably packaged in lyophilized and hence stable form, the pharmaceutical compositions advantageously comprise substances stabilizing the growth factor in the active form. Such stabilizers inhibit the formation of intermolecular disulfide bonds, thereby preventing the polymerization of the active substance. However, the amount of stabilizer should be carefully measured in order to concomitantly prevent the reduction of the active substance to the inactive monomeric form. Examples of such substances are: Cystein, Cysteamine, or glutathione in reduced form.

Non-limiting examples of oils include fats and oils such as olive oil and hydrogenated oils; waxes such as beeswax and lanolin; hydrocarbons such as liquid paraffin, ceresin, and squalene; fatty acids such as stearic acid and oleic acid; alcohols such as cetyl alcohol, stearyl alcohol, lanolin alcohol, and hexadecanol; and esters such as isopropyl myristate, isopropyl palmitate and butyl stearate. As examples of surfactants there may be cited anionic surfactants such as sodium stearite, sodium cetylsulfate, polyoxyethylene laurylether phosphate, sodium N-acyl glutamate; cationic surfactants such as stearyldimethylbenzylammonium chloride and stearyltrimethylammonium chloride; ampholytic surfactants such as alkylaminoethylglycine hydrochloride solutions and lecithin; and nonionic surfactants such as glycerin monostearate, sorbitan monostearate, sucrose fatty acid esters, propylene glycol monostearate, polyoxyethylene oleylether, polyethylene glycol monostearate, polyoxyethylene sorbitan monopalmitate, polyoxyethylene coconut fatty acid monoethanolamide, polyoxypropylene glycol (e.g. the materials sold under the trademark “Pluronic”), polyoxyethylene castor oil, and polyoxyethylene lanolin. Examples of humectants include glycerin, 1,3-butylene glycol, and propylene glycol; examples of lower alcohols include ethanol and isopropanol; examples of thickening agents include xanthan gum, hydroxypropyl cellulose, hydroxypropyl methyl cellulose, polyethylene glycol and sodium carboxymethyl cellulose; examples of antioxidants include butylated hydroxytoluene, butylated hydroxyanisole, propyl gallate, citric acid and ethoxyquin; examples of chelating agents include disodium edetate and ethanehydroxy diphosphate; examples of buffers include citric acid, sodium citrate, boric acid, borax, and disodium hydrogen phosphate; and examples of preservatives are methyl parahydroxybenzoate, ethyl parahydroxybenzoate, dehydroacetic acid, salicylic acid and benzoic acid. These substances are merely exemplary, and those of skill in the art will recognize that other substances may be substituted with no loss of functionality.

### EXAMPLES

### Example 1

### Relieving the Symptoms of Nickel Allergy

A subject between 40 and 50 years of age, diagnosed with nickel allergy by dermatologist experienced repeated allergic contact dermatitis on her skin, manifesting itself with scaling of the skin and small lacerations under the scales. The scales and lacerations were primarily under her feet and on her fingertips causing the subject pain when walking and even disturbing sleep and rest due to pain in her fingers. The subject had tried all therapies recommended for her condition by her dermatologist, without noticeable success.

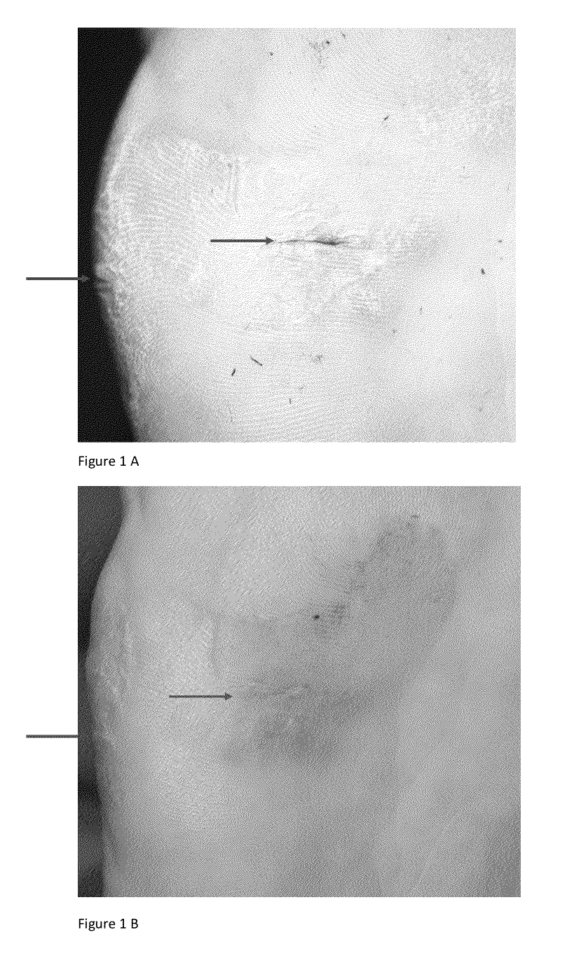

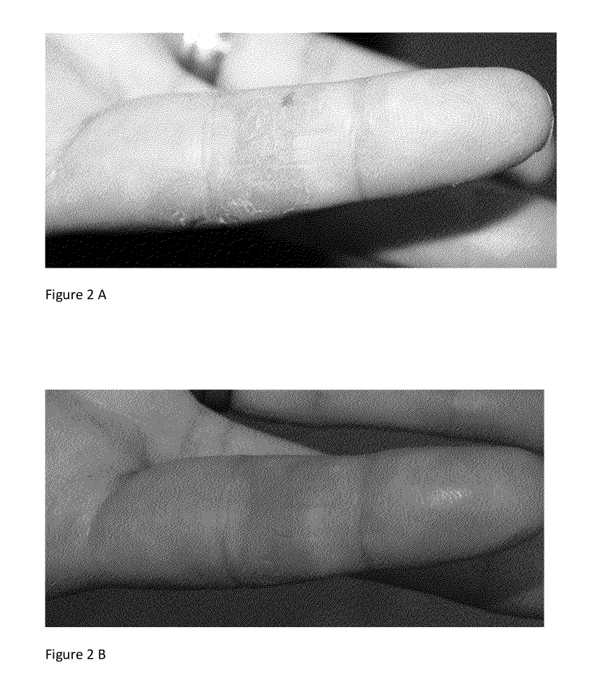

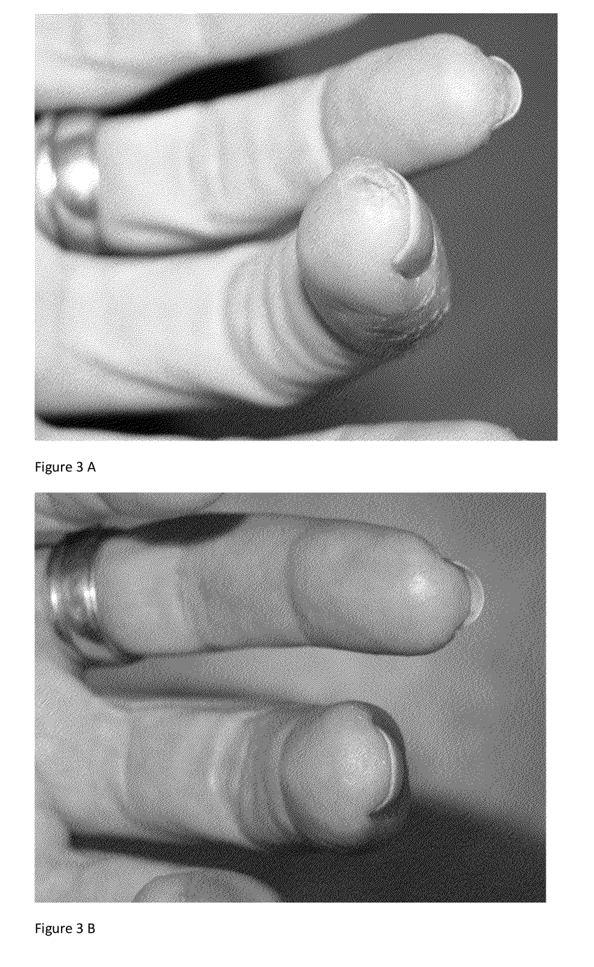

The subject applied an EGF growth factor-containing glycerol based composition, containing purified water and hyaluronate (0.1%). The growth factor is present in the composition as 0.0005%, of the present invention by spreading 3-4 drops of the composition as a thin layer on her hands and feet, particularly the scaled areas, twice a day for 13 days except on days 6 and 9. The subject kept all activities constant, such as consumption of food, and daily activities. The improvement was such that on day six she was to large extent free from pain in the scale areas of the feet (FIG. 1A (before treatment) and B (after 6 day treatment)). After 13 days the subject was more or less pain free and the scales and lacerations had mostly disappeared (FIGS. 2 A and B) and 3 A and B)). The subject was not bothered by her condition at all anymore and described the effect of the composition of the present invention containing Epidermal growth factor as “life changing”.

### Example 2

### Relieving the Symptoms of Neurodermatitis (Lichen Simplex Chronicus)

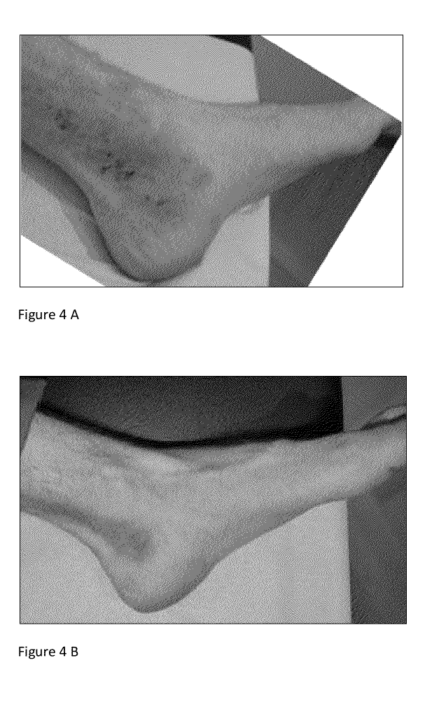

A subject 30 years old had suffered from Neurodermatitis since childhood on an ankle. The ankle region had thickened skin and itchy patches within a larger purple area. The subject had used many topical medications under guidance of medical doctors since childhood with limited success. A two drops of a composition according to the present invention containing an Epidermal growth factor (0.00025% w/w) in glycerol/purified water solution with CaCl₂ (1 mM), were applied to the ankle area twice a day for four weeks. Before the treatment the state of the affected ankle was documented by a Medical doctor with picture (FIG. 4 A). After four weeks of treatment with a growth factor containing composition the itchiness had resided and the affected area had decreased 20 fold in size (FIG. 4 B). According to a Medical doctor the subject had suffered from Neurodermatitis from childhood and never used a topical medication with such a great result.

### Example 3

### Alopecia Greata and Folliculitis Relief

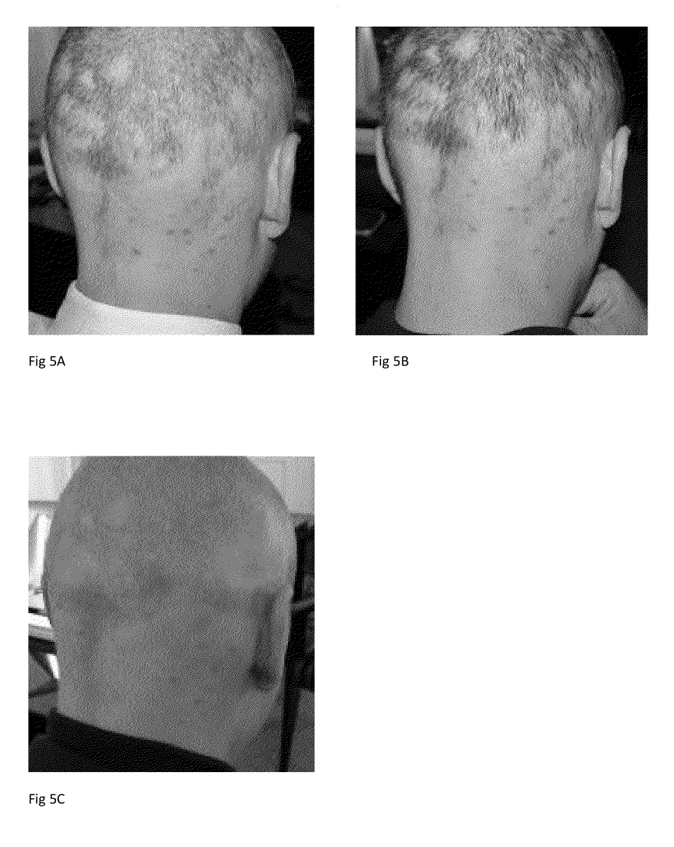

Folliculitis is inflammation of the hair follicles, that may be caused by bacteria, fungal- or viral infection. Subject, a 33 old male suffered from Folliculitis with severe itchiness and Alopecia greata (spot baldness) in the scalp (FIG. 5A). Under the supervision of a doctor the subject treated the itchy areas and the Alopecia areas with a growth factor containing composition of the present invention (Epidermal growth factor (0.0005% w/w) in glycerol/purified water solution with 0.7% w/w ethanol). After one week the itchiness had stopped and a significant improvement of the Folliculitis was observed (FIG. 5B). After 4 weeks the exanthema (rash) had disappeared. After 12 weeks of treatment (FIG. 5C) the itchiness and the exanthema were absent and the alopecia spots were still present but the alopecia was not active. The subject experienced significant improvement in quality of life.

### Example 4

### Relieving the Symptoms of Acne

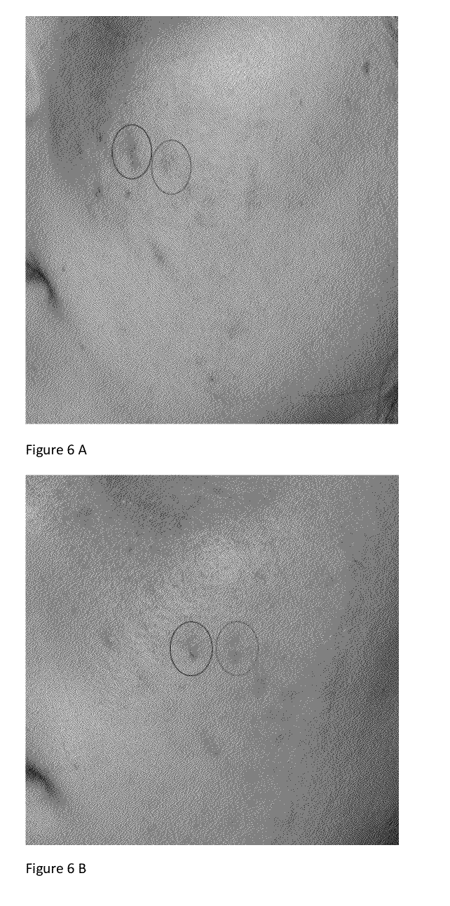

A female 21 years old subject was diagnosed with mild to moderate acne that had troubled her for 5-6 years. Dermatologists had prescribed topical and oral treatments with limited success. The condition of the subject was monitored by a medical doctor and documented with pictures and before and after a 14 day treatment (FIGS. 6 A Day 1 and 6 B Day 14, respectively) with a growth factor containing composition of the invention (Epidermal growth factor (0.00025% w/w) in glycerol/purified water solution with hyaluronate (0.2% w/w), CaCl2 (1 mM)). Daily application of 2 drops of the composition was administered in the evening. The treatment resulted in improved skin condition and particularly lower inflammation of the skin. Subjects that used the growth factor containing composition of the invention periodically over longer periods of time reported how the acne subsided by the treatment and they observed less scarring of the skin.

### REFERENCES

Ito I, Fixman E D, Asai K, Yoshida M, Gounni A S, Martin J G, Hamid Q. “Platelet-derived growth factor and transforming growth factor-beta modulate the expression of matrix metalloproteinases and migratory function of human airway smooth muscle cells.” Clin Exp Allergy. 2009 September; 39(9):1370-80. Epub 2009 Jun. 11.
Nakatani Y, Tanimoto K, Tanaka N, Tanne Y, Kamiya T, Kunimatsu R, Tanaka E, Tanne K. “Effects of hyaluronan oligosaccharide on the expression of MMP-1 in periodontal ligamentcells.” Arch Oral Biol. 2009 August; 54(8):757-63. Epub 2009 Jun. 11.
Technical specification sheet, EGF Recombinant Human Epidermal Growth Factor, Cell Sciences, MA, USA. (http://www.cellsciences.com/PDF/CRE100.pdf)
5.1.3 Efficacy of Antimicrobial Preservation: 528-529, European Pharmacopoeia 6.0.

1. A pharmaceutical composition comprising at least one growth factor and at least one pharmaceutically acceptable excipient for treating of at least one of the conditions or diseases select from the group consisting of: challenged skin due to systemic lupus erythematosus, Lichen simplex chronicus, blepharitis and blepharokeratoconjuctivitis, symptoms of nickel allergy that may cause or aggravate abnormal skin conditions manifested as blisters, scaling of skin and/or lacerations; stretch marks, acne, abnormal pigmentation of the skin, melasma or chloasma and postinflammatory hyperpigmentation, and to provide positive effect on challenged or damaged skin that has been exposed to irritating or damaging conditions due to radiation, or exposure to chemicals resulting in photosensitation of the skin, such as psoralens and furocoumarins.

2. The composition of claim 1 comprising an excipient formula that stabilizes the growth factor protein.

3. The composition of claim 1 where the growth factor is a heterologous growth factor isolated from a host organism or host cells expressing the respective heterologous gene encoding said heterologous growth factor.

4. The composition of claim 3 where the heterologous growth factor has been produced, using recombinant gene technology, in a host organism or host cell selected from the group consisting of bacteria, yeast, fungi, insect cells, an animal or animal cells, a plant or plant cells, either stably transformed or by transient expression technology.

5. The composition of claim 3, where the heterologous growth factor is a purified heterologous growth factor.

6. The composition of claim 3 where the purified heterologous growth factor used in the composition is purified to a high level of purification in the range of about 90-99.9%

7. The composition of claim 3 where the purified heterologous growth factor used in the composition has been purified to a purification level in the range of about 70-90% pure

8. The composition of claim 3 where the heterologous growth factor used in the composition is partially purified in the range of about 1-70%, in an extract.

9. The composition of claim 2, wherein said protein stabilizing formula comprises glycerol, water, and calcium chloride.

10. The composition of claim 1, wherein said protein stabilizing formula comprises the following ingredients (% by weight): glycerol in the range from 10 to 90%, calcium chloride in the range from 0.1 mM to 200 mM, buffered in the pH range of 6-9 and purified water q.s.

11. The composition of claim 1 comprising hyaluronic acid.

12. The composition of claim 11 wherein the hyaluronic acid is provided in a concentration in the range of about 0.01 to about 2% by weight.

13. The composition of any of claim 1 wherein the growth factor is provided as a component of a transgenic plant extract comprised in the composition.

14. The composition of claim 13 where the growth factor is present in the transgenic plant extract in amount in the range of about 0.0001% to about 70% of the total protein content.

15. The composition of claim 13, wherein said plant extract has been purified to enrich for said growth factor.

16. The composition of claim 1, comprising more than one growth factor.

17. The composition of claim 16, where said more than one growth factor are present as components of a mixture of extracts.

18. The composition of claim 17, where said more than one growth factor are present in the mixture of extracts in an amount in the range of about 0.0001% to about 70% of the protein content.

19. The composition of claim 1, wherein the at least one growth factor or more than one growth factors is isolated and purified from transgenic plants to a level of purity in the range of about 70% to about 99.9%.

20. The composition of claim 1, wherein one or more purified growth factors are added to a growth factor containing extract.

21. The composition of claim 1, wherein the at least one growth factor or more than one growth factor is selected from the group consisting of Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g), Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2, BMP-3, BMP-4, BMP-5, BMP-6, BMP-7, BMP-8a), Hepatocyte Growth Factor (HGF), Leptin, Noggin, and Thymosin beta 4.

22. The composition of claim 1, wherein said growth factor or cytokine originates from corresponding human gene sequence.

23. The composition of claim 1, wherein said growth factor or cytokine originates from synthesized gene corresponding to human gene sequence for respective growth factor or cytokine.

24. The composition of claim 1, wherein the transgenic plant extract contains dehydrins and/or globulins or other seed proteins.

25. The composition of claim 1, wherein the composition is in the form selected from creams, lotions, gels, dressings, shampoos, tinctures, pastes, ointments, salves, powders, liquid or semiliquid formulations, serums, patches, liposomal preparations, solutions, suspensions, liposome suspensions, W/O or O/W emulsions, ointments, pomades and pastes and a skin softener cream, a facial pack, a massage cream, and a nutrient cream or a nutrient emulsion.

26. A pharmaceutical composition comprising a growth factor and an antifungal agent.

27. The composition of claim 26, wherein the antifungal agent is selected from the group of polyene antifungals, imidazole, triazole, thiazole antifungals and allylamin.

28. The composition of claim 26, wherein the antifungal agent is selected from Clotrimazole, Terbinafine, Miconazole, Ketoconazole, Amorolfine and Econazole.

29. The composition of claim 2 further comprising an antifungal agent.

30. A growth factor for use as a medicament for treatment of a skin condition selected from one or more of challenged skin due to systemic lupus erythematosus, Lichen simplex chronicus, blepharitis and blepharokeratoconjuctivitis, symptoms of nickel allergy that may cause or aggravate abnormal skin conditions manifested as blisters, scaling of skin and/or lacerations, abnormal pigmentation of the skin; melasma or chloasma and postinflammatory hyperpigmentation and to provide positive effect on challenged or damaged skin that has been exposed to irritating or damaging conditions due to radiation, or exposed to chemicals resulting in photosensitation of the skin, such as psoralens and furocoumarins.

31. The growth factor of claim 30, selected from the group consisting of Epidermal Growth Factor (EGF), Vascular Epithelial Growth Factor (VEGF), Platelet-Derived Growth Factor (PDGF) including PDGF-AA, PDGF-BB, and PDGF-Rb, Fibroblast Growth Factors (FGFs) including FGF-a, and FGF-b FGF-4 and FGF-6, Transforming Growth Factors-beta (TGFs-b) including TGF beta-1, TGF beta-2, TGF beta-3, Transforming Growth Factor-alpha (TGF-a), Erythropoietin (Epo), Insulin-Like Growth Factor-I (IGF-I), Insulin-Like Growth Factor-II (IGF-II), Interleukin-1 (IL-1) including IL-1 alpha and IL-1 beta, Interleukin-2 (IL-2), Interleukin-4 (IL-4), Interleukin 5 (IL-5), Interleukin-6 (IL-6), Interleukin-7 (IL-7), Interleukin-8 (IL-8), Interleukin-10 (IL-10), Interleukin-13 (IL-13), Interleukin-15 (IL-15), Interleukin-18 (IL-18), Interleukin-20 (IL-20), Tumor Necrosis Factor-alpha (TNF-a), Tumor Necrosis Factor-beta (TNF-b), Interferon-gamma (INF-g), Granulocyte Colony Stimulating Factor (G-CSF), Granulocyte Macrophage Colony Stimulating Factor (GM-CSF), Macrophage Colony stimulating factor (M-CSF), FLT-3 ligand, Heparin binding-EGF (Hb-EGF), Leukemia inhibiting factor (LIF), Stem cell factor (SCF), Placenta Growth Factor (PLGF), Nerve Growth Factor (NGF), Keratinocyte Growth Factor (KGF), Bone morphogenesis Proteins (BMPs; BMP-2, BMP-3, BMP-4, BMP-5, BMP-6, BMP-7, BMP-8a), Hepatocyte Growth Factor (HGF), Leptin, Noggin, and Thymosin beta 4.

32. The growth factor of claim 30, which is Epidermal Growth Factor.

33. The growth factor of claim 30, which is a heterologous growth factor produced in a transgenic host organism or cell.

34. The growth factor of claim 33, which is produced in a host organism or cell selected from plant, bacteria, yeast, fungi, animal or animal cell, including insect cells.

35. The growth factor of claim 30, which is a human growth factor.
